(19) United States
(12) Patent Application Publication
(10) Pub. No.: US 2010/0068259 A1
(43) Pub. Date: Mar. 18, 2010

(54) NATURAL PRODUCT INHIBITORS OF 3DG

(76) Inventors: Annette Tobia, Wyndmoor, PA (US); Marcy Alice, Doylestown, PA (US); Bangying Su, Elkins Park, PA (US); Takeshi Niwa, Osaka (JP)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/474,904

(22) Filed: May 29, 2009

Related U.S. Application Data
(60) Provisional application No. 61/057,398, filed on May 30, 2008.

(51) Int. Cl. A61K 9/127; A61K 35/56; A61K 36/00; A61K 38/16; A61K 36/73; A61K 36/87; A61P 17/00; A61K 36/31; A61K 35/12; A61K 36/8962; A61K 36/42; A61K 36/736; A61K 36/45; A61K 36/534; A61K 36/16; A61K 36/82; A61K 36/258; A23L 2/84; A23F 3/20

(52) U.S. Cl. 424/450; 424/547; 424/769; 514/12; 424/765; 424/725; 424/766; 424/776; 424/755; 424/520; 424/754; 424/758; 424/735; 424/732; 424/747; 424/752; 424/729; 424/728; 424/773; 424/59; 426/10

(57) ABSTRACT

Compositions are disclosed which have as a component thereof an inhibitor of the enzymatic production of 3-deoxyglucosone (3DG) from fructoselysine and/or an inactivator of 3DG, and which are useful for the treatment or prophylaxis of a condition or disease state that is alleviated by inhibiting such 3DG production. Methods of using such compositions, e.g., for improving the appearance, texture and/or elasticity of aging skin, are also disclosed.

### CROSS-REFERENCE TO RELATED APPLICATIONS

The present application claims the benefit of U.S. Provisional Patent Application No. 61/057,398, filed May 30, 2008, the entire disclosure of which is incorporated by reference herein.

### BACKGROUND

The amino acid lysine is an essential amino acid in mammals, and a biochemical pathway exists to recover lysine so that it can be reused. U.S. Pat. No. 6,004,958 to Brown et al. discloses that lysine is enzymatically recovered from fructoselysine (FL) with the concomitant production of 3-deoxyglucosone (3DG) in the Amadori Pathway. 3DG and the enzyme are also found in skin, as disclosed in International Publication No. WO 03/089601. Lysine becomes glycated in the body as a result of a reversible reaction between glucose and the ε-NH2 groups of lysine-containing proteins. This process proceeds via a Schiff base intermediate which rearranges to the more stable FL, an “Amadori product.” Cooked animal products introduced by diet can also contribute glycated protein. Glycated protein is eventually degraded resulting in FL. Fructoseamine-3-Kinase (F3K) phosphorylates FL on its 3′-OH creating fructoselysine-3-phosphate (FL3P) which then spontaneously decomposes into lysine, Pi, and 3DG. Thus F3K allows the body to recover lysine, however, this process causes the production of 3DG, a highly reactive dialdehyde molecule. 3DG has been shown to chemically interact with protein lysine residues, in an early, irreversible step in the process of forming protein cross-links that are characteristic of advanced glycation end products (AGEs).

U.S. Pat. No. 6,004,958 to Brown et al. and International Publication Application No. WO 03/089601 describe a class of compounds which inhibit the enzymatic conversion of FL to FL3P, inhibit the formation of lysine from the deglycation of FL, inhibit the formation of 3DG, as well as provide for the inactivation of 3DG and detoxification of 3DG. Specific compounds which are representative of the class have also been described (Brown et al., International Publication No. WO 98/33492). For example, it was found that urinary or plasma 3DG can be reduced by meglumine, sorbitollysine, mannitollysine, and galactitollysine. Id. It was also found that diets high in glycated protein are harmful to the kidney and cause a decrease in birth rate. Id. It has also been disclosed that the FL pathway is involved in kidney carcinogenesis. Id. Further, previous studies demonstrate that diet and 3DG can play a role in carcinogenesis associated with this pathway (see International Publication Nos. WO 00/24405; WO 00/62626; WO 98/33492).

3DG is a highly reactive molecule that can be detoxified in the body by at least two pathways. In one pathway, 3DG is reduced to 3-deoxyfructose (3DF) by aldehyde reductase, and the 3DF is then efficiently excreted in urine (Takahashi et al., 1995, Biochemistry 34:1433-8). Another detoxification reaction oxidizes 3DG to 3-deoxy-2-ketogluconic acid (DGA) by oxoaldehyde dehydrogenase (Fujii et al., 1995, Biochem. Biophys. Res. Commun. 210:852-7).

Results of studies to date show that one of these enzymes, aldehyde reductase, is adversely affected in diabetes. When isolated from diabetic rat liver, this enzyme is glycated on lysine at positions 67, 84 and 140 and has a low catalytic efficiency when compared with the normal, unmodified enzyme (Takahashi et al., 1995, Biochemistry 34:1433-8). Since diabetic patients have higher ratios of glycated proteins than normoglycemic individuals, they have higher levels of 3DG, which at once tends to inactivate aldehyde reductase and reduces the enzyme's ability to detoxify this reactive molecule by reduction to 3DF. There is supportive evidence that this detoxification of 3DG to 3DF is impaired in diabetic humans since their ratio of urinary and plasma 3DG to 3DF differs significantly from non-diabetic individuals (Lal et al., 1997, Arch. Biochem. Biophys. 342:254-60). Overexpression of aldehyde reductase protects PC 12 cells from the cytotoxic effects of methylglyoxal or 3DG (Suzuki et al., 1998, J. Biochem. 123:353-7).

The mechanism by which aldehyde reductase works has been studied. These studies demonstrated that this important detoxification enzyme is inhibited by aldose reductase inhibitors (ARIs) (Barski et al., 1995, Biochemistry 34:11264-75). ARIs are currently under clinical investigation for their potential to reduce certain diabetic complications. These compounds, as a class, have shown some effect on short-term diabetic complications, but they lack clinical effect on long-term diabetic complications and they worsen kidney function in rats fed a high protein diet. This finding is consistent with the newly discovered metabolic pathway for lysine recovery.

Aminoguanidine (AG), an agent that detoxifies 3DG pharmacologically via formation of rapidly excreted covalent derivatives (Hirsch et al., 1992, Carbohydr. Res. 232:125-30), reduces AGEs-associated retinal, neural, arterial, and renal pathologies in animal models (Brownlee, 1994, Diabetes 43:836-41; Brownlee et al., 1986, Science 232:1629-32; Ellis et al., 1991, Metabolism 40:1016-9; Soulis-Liparota et al., 1991, Diabetes 40:1328-34, and Edelstein et al., 1992, Diabetologia 35:96-7).

Past studies have concentrated on the role of 3DG in diabetes. Diabetic humans have elevated levels of 3DG and 3DF, 3DG's detoxification product, in plasma (Niwa et al., 1993, Biochem. Biophys. Res. Commun. 196:837-43; Wells-Knecht et al., 1994, Diabetes 43:1152-6) and in urine (Wells-Knecht et al., 1994, Diabetes 43:1152-6), as compared with non-diabetic individuals. Furthermore, diabetics with nephropathy were found to have elevated plasma levels of 3DG compared to non-diabetics (Niwa et al., 1993, Biochem. Biophys. Res. Commun. 196:837-43).

A recent study comparing patients with insulin-dependent diabetes mellitus (IDDM) and noninsulin-dependent diabetes mellitus (NIDDM) confirmed that 3DG and 3DF levels were elevated in blood and urine from both types of patient populations. Thus the normal pathway for reductive detoxification of 3DG (conversion to 3DF) may be impaired in diabetic humans (Lal et al., 1995, Arch. Biochem. Biophys. 318:191-9). It has even been shown that incubation of glucose and proteins in vitro under physiological conditions produces 3DG.

In turn, it has been demonstrated that 3DG glycates and crosslinks protein creating detectable AGE products (Baynes et al., 1984, Methods Enzymol. 106:88-98; Dyer et al., 1991, J. Biol. Chem. 266:11654-60).

Furthermore, elevated levels of 3DG-modified proteins have been found in diabetic rat kidneys compared to control rat kidneys (Niwa et al., 1997, J. Clin. Invest. 99:1272-80). 3DG has the ability to inactivate enzymes such as glutathione reductase, a central antioxidant enzyme. It has also been shown that hemoglobin-AGE levels are elevated in diabetic individuals (Makita et al., 1992, Science 258:651-3), and other AGE proteins have been shown in experimental models to accumulate with time, increasing from 5-50 fold over periods of 5-20 weeks in the retina, lens and renal cortex of diabetic rats (Brownlee, 1994, Diabetes 43:836-41). In addition, 3DG is a teratogenic factor in diabetic embryopathy leading to embryo malformation (Eriksson et al., 1998, Diabetes 47:1960-6). This appears to arise from 3DG accumulation, which leads to superoxide-mediated embryopathy.

Nonenzymatic glycation, in which reducing sugars are covalently attached to free amino groups and ultimately form AGEs, occurs during normal aging and is accelerated in diabetes mellitus (Bierhaus et al., 1998, Cardiovasc. Res. 37:586-600). Crosslinking of proteins and the subsequent AGEs formation are irreversible processes that alter the structural and functional properties of proteins, lipid components, and nucleic acids (Bierhaus et al., 1998, Cardiovasc. Res. 37:586-600). These processes are believed to contribute to the development of a range of diabetic complications including nephropathy, retinopathy, and neuropathy (Rahbar et al., 1999, Biochem. Biophys. Res. Commun. 262:651-6).

Inhibition of AGE formation reduced the extent of nephropathy in diabetic rats (Ninomiya et al., 2001, Diabetes 50:A178-179). Therefore, substances that inhibit AGE formation and/or oxidative stress appear to limit the progression of diabetic complications and may offer new approaches for therapeutic interventions in the treatment of diabetes (Thomalley, 1996, Endocrinol. Metab. 3:149-166; Bierhaus et al., 1998, Cardiovasc. Res. 37:586-600).

Hemoglobin-AGE levels are elevated in diabetic individuals (Makita et al., 1992, Science 258:651-3), and other AGE proteins have been shown in experimental models to accumulate with time, increasing from 5-50 fold over periods of 5-20 weeks in the retina, lens and renal cortex of diabetic rats (Brownlee, 1994, Diabetes 43:836-41).

3DG induces reactive oxygen species in human umbilical vein endothelial cells, which results in oxidative DNA damage (Shimoi et al., 2001, Mutat. Res. 480-481:371-8). Additionally, 3DG-induced reactive oxygen species contribute to the development of diabetic complications (araki, 1997, Nippon Ronen Igakkai Zasshi 34:716-20). Specifically, 3DG induces heparin-binding epidermal growth factor, a smooth muscle mitogen that is abundant in atherosclerotic plaques. This suggests that an increase in 3DG may trigger atherogenesis in diabetes (Taniguchi et al., 1996, Diabetes 45 Suppl. 3:S81-3; Che et al., 1997, J. Biol. Chem. 272:18453-9).

Finally, a direct link between serum levels of 3DG in diabetics and the risk of development of diabetic complications has been demonstrated (Kusunoki et al., 2003, Diabetes Care 26:1889-94). The results show that the fasting serum 3DG level is elevated in diabetic patients and that the patients with relatively higher 3DG levels were prone to suffer from more severe complications, indicating a possible association of 3DG with diabetic microangiopathy.

3DG also produces harmful effects unrelated to diabetes. For example, it was demonstrated that 3DG induces apoptosis in macrophage-derived cell lines (Okado et al., 1996, Biochem. Biophys. Res. Commun. 225:219-24), and is toxic to cultured cortical neurons (Kikuchi et al., 1999, J. Neurosci. Res. 57:280-9) and PC12 cells (Suzuki et al., 1998, J. Biochem. 123:353-7). A recent study on the cause of amyotropic lateral sclerosis, a form of motor neuron disease, has suggested that accumulation of 3DG can lead to neurotoxicity as a result of ROS generation (Shinpo et al., 2000, Brain Res. 861:151-9).

Previous studies demonstrated that 3DG glycates and crosslinks protein leading to the complex mixture of compounds known as AGEs (Baynes et al., 1984, Methods Enzymol. 106: 88-98; Dyer et al., 1991, J. Biol. Chem. 266:11654-60). AGEs have been implicated in most inflammatory diseases such as atherosclerosis and dementia, as well as diabetes. They are most commonly formed on long-lived structural proteins such as collagen.

AGEs have specific cell receptors commonly referred to as RAGE. The activation of cellular RAGE on endothelium, mononuclear phagocytes, and lymphocytes triggers the generation of free radicals and the expression of inflammatory gene mediators (Hofmann et al., 1999, Cell 97:889-901). This increased oxidative stress leads to the activation of the transcription factor NF-kB and promotes the expression of NF-kB genes that have been associated with atherosclerosis (Bierhaus et al., 1998, Cardiovasc. Res. 37:586-600).

In relationship to cancer, blockage of RAGE activation inhibits several mechanisms linked to tumor proliferation and trans-endothelial migration of tumor cells. This also decreases growth and metastases of both spontaneous and implanted tumors (Taguchi et al., 2000, Nature 405:354-60).

AGEs arise from normal metabolism and are the products of the reaction of non-reducing sugars with amino groups of protein, lipid or nucleic acid. AGEs can be introduced in foods by various ingredient combinations and cooking. Foods high in AGEs include those that are cooked at high temperature such as broiling, grilling, frying and roasting (Goldberg et al., 2004, J Am Diet Assoc 104:1287-1291). A portion of ingested AGEs are absorbed and appear in the circulation (Koschinsky et al., 1997, Proc Natl Acad Sci USA 94:6474-6497). Small AGE-modified peptides can pass through the intestinal epithelium (Huebschmann et al., 2006, Diabetes Care 29:1420-1432). A diet rich in glycated protein results in increased circulating AGE products (Uribarri et al. 2005. Ann NY Acad Sci 1043:461-466).

Circulating AGEs levels are also dependent on environmental factors and physiological state. Plasma AGE levels are increased in people with diabetes due to increased glucose levels or in patients with renal failure due to decreased clearance by the kidneys (Odani et al. 1999, J. Chromatogr B 731:131-140; Odani et al., Biochem Biophys res Commun 256:89-93). Tobacco smokers have higher circulating levels of AGEs (Cerami et al., 1997. Proc Natl Acad Sci USA 94:13915-20).

Ingestion of dietary AGEs correlates with circulating AGEs, and these in turn correlate with markers of inflammation and oxidative stress (Koschinsky et al., 1997, Proc Natl Acad Sci USA 94:6474-6497; Vlassara et al., 2002, Proc Natl Acad Sci USA 99:15596-15601; Uribarri et al., 2007. J. Gerontol A Biol Sci Med Sci 62:427). Mice kept on a low AGE diet showed reduced AGE accumulation, reduced oxidative stress, and increased lifespan (Cai et al., 2000, Am J Pathol 170:1893). Diabetic patients fed a high AGE meal show increased levels of AGE in the serum, increased oxidative stress, and impairment of vascular function (Negrean et al. 2007. Am J Clin Nutr 85:1236-43). Diabetic mice fed a high AGE diet show impaired wound healing compared to animals on a low AGE diet (Peppa et al., 2003. Diabetes 52:2805-13). Absorption of one AGE product, carboxymethylysine, by the oral adsorbent agent AST-120 reduced AGE levels in nondiabetic patients with chronic renal failure (Ueda et al. 2006. Mol Med 12:180-184).

Due to the detrimental effect of circulating 3DG, it is desirable to decrease 3DG exposure by minimizing ingestion of 3DG from food or nutritional supplements. As 3DG has detrimental effects on skin cells, it is also desirable to decrease 3DG exposure on the skin by decreasing its concentration in topical preparations or cosmetics. 3DG can be enzymatically reduced to 3DF by aldehyde reductase (Kato et al., 1990, Biochim Biophys Acta 1035:71-76; Liang et al., 1991, Eur J Biochem 197:373-379; Knecht et al., 1992, Arch Biochem Biophys 294:130-137; Niwa 1999, J Chromatog B Biomed Sci Appl 731:23-36). 3DF is then efficiently excreted in urine (Takahashi et al., 1995, Biochemistry 34:1433-8). 3DG can be chemically inactivated with aminoguanidine, cysteine or pyridoxal 5′-phosphate (Nakamura and Niwa, 2005, J Am Soc Nephrol, 16:144-150; Igaki et al., 1990, Clin Chem 36:631-634).

It has been observed that certain agents having the F3K inhibitory activity are also effective for the treatment or prevention of a condition known as “dry eye” (keratitis sicca). See U.S. Provisional Application 61/043,162 filed Apr. 8, 2008. Dry eye is a chronic dryness of the corneal and conjunctivial surfaces and results from a decrease in the production of tear components or from an altered ratio of the individual oil, water and mucus components of the tear film which moistens the eye. The condition is manifested by a variety of symptoms including redness, soreness, burning and itching of the eye, photophobia, blurred vision, foreign body sensation and contact lens intolerance. It is believed that the aforementioned agents promote moistening of corneal and conjunctivial surfaces due to enhanced mucus production resulting from an increase in goblet cells, which are a primary source of excreted mucin. In that goblet cells are present in other tissues (digestive and respiratory epithelia), agents that increase mucin production may have additional utilities in treating conditions such as dry mouth (xerostomia) and constipation.

### SUMMARY OF THE INVENTION

As mentioned above, it was previously reported that the action of F3K in the lysine recovery pathway leads to the production of highly reactive 3DG, which has an essential role in the formation of AGEs.

It has now been discovered, in accordance with the present invention, that a wide range of natural products comprise one or more components that inhibit the enzymatic conversion of FL to FL3P and/or inactivate 3DG. This discovery can be put to practical use in the following ways:
a method for the treatment or prophylaxis of a condition or disease state which is alleviated by inhibiting the enzymatic conversion of FL to FL3P, in a patient in need of such treatment or prophylaxis, by administering to the patient at least one natural product having as a component thereof an inhibitor of the enzymatic conversion of FL to FL3P, in an amount effective to inhibit such conversion;
a method of preventing, ameliorating and/or reversing the intrinsic and/or extrinsic aging of skin, by topically applying to aging skin a composition comprising at least one natural product having as a component thereof an inhibitor of the enzymatic conversion of FL to FL3P, in an amount effective to inhibit such conversion;
a method of improving the appearance, texture, or elasticity of aging skin, by topically applying to aging skin a composition comprising at least one natural product having as a component thereof an inhibitor of the enzymatic conversion of FL to FL3P, in an amount effective to inhibit such conversion; or
a method of treating skin damage due to oxidative stress and/or the production of AGEs, by topically applying to damaged skin a composition comprising a natural product having as a component thereof an inhibitor of the enzymatic conversion of FL to FL3P, in an amount effective to inhibit such conversion.

It has also been discovered, in accordance with this invention, that numerous natural products contain varying amounts of 3DG, which in certain instances may pose a health risk to subjects that use them, both humans and animals. The purity of such 3DG-containing substances, when used as food, cosmetic, pharmaceutical or dietary supplement ingredients, can be enhanced (and the health risk potential correspondingly diminished) by reducing the 3DG content thereof, for example, by admixture with a 3DG inactivating agent. Suitable inactivating agents for this purpose are identified in the following detailed description.

### DETAILED DESCRIPTION OF THE INVENTION

Natural products that contain an inhibitor of the F3K enzyme and/or a 3DG inactivator may be used to advantage for treating or preventing conditions or disease states that are linked to 3DG which is produced as a by-product of F3K activity. The disease states that may be treated or prevented by the methods of the invention include inflammatory disorders, complications of diabetes, diseases of aging, hypertension, stroke, neurodegenerative disorders, circulatory disease, atherosclerosis, osteoarthritis and cataracts. The method described herein may also be used for the treatment or prophylaxis of skin conditions, particularly those associated with intrinsic or extrinsic aging. Intrinsic aging of the skin is the gradual deterioration that results from the normal aging process, which produces change in the chemical structure of proteins, including collagen and elastin, due, in part, to the formation of AGEs. A number of extrinsic factors, often acting in conjunction with the normal aging process, cause premature aging of the skin. Most extrinsic aging is brought about by sun exposure or “photoaging”; however, other factors, such as repetitive facial expressions and smoking can contribute to such premature aging.

A variety of different natural products can be used for treating or preventing a condition or disease state that is alleviated by inhibiting the enzymatic conversion of fructoselysine to fructoselysine-3-phosphate and/or by inactivating 3DG in vivo. The term “natural product”, as used herein, refers to a chemical substance found in nature, such as a substance obtained from tissues of terrestrial plants, marine animals or plants, and other living organisms, as well as derivatives of such substances. Representative examples of natural products (and extracts thereof) which may be used in the practice of this invention include materials of plant and animal origin, polypeptides, oligopeptides, vitamins, provitamins and the like. Natural product extracts are commercially available from various sources and may be prepared using the extraction methods generally described in U.S. Pat. No. 6,485,756 to Aust and Wilmott.

Natural products suitable for practicing this invention can be identified using the F3K assay described hereinbelow. The results of performing this assay on a wide range of natural products are as set forth in Tables 1 and 1A, below. Alternative assays for determining F3K inhibitory activity, by direct measurement of fructoselysine-3-phosphate production, are described in the aforementioned U.S. Pat. No. 6,004,958.

Supplemental active agents may be administered in conjunction with the natural products described herein, if desired. Suitable supplemental active agents include, by way of example, anesthetics, antibiotics, anti-allergenics, anti-fungals, antiseptics, anti-irritants, anti-inflammatory agents, anti-microbials, analgesics and anti-hypertensive agents, e.g., ACE inhibitors.

The natural products described herein, along with any supplemental active agent(s), may be administered using any amount and any route of administration effective to inhibit enzymatic 3DG production. The exact amount to be administered may vary depending on the species, age, and general condition of the patient, the nature of the condition or disease state being treated, the specific natural product used and its mode of administration. As used herein, the term “patient” refers to animals, including mammals, preferably humans and domestic animals.

The effectiveness of the amount of natural product administered to a patient can be assessed by feeding to the patient, either human or animal, a food rich in glycated lysine residues or FL and measuring the amount of 3DG and 3DF in their urine, both before and after feeding. Patients that have an effective inhibitory amount of F3K inhibitor in their systems will exhibit decreased secretion of both 3DG and/or 3DF and increased urinary secretion of FL, as compared to levels secreted by the same patients prior to administration of the natural product(s). The natural products used in the practice of this invention are commonly available in powder form. As such, they may readily be formulated for topical or oral administration, topical administration being preferred.

Topical formulations including any of various dermatologically acceptable excipients may be prepared in the form of an emulsion, a cream, a balm, a gloss, a lotion, a salve, a mask, a serum, a toner, an ointment, an oil, a mousse, a gel, a pomade, a solution, a liquid spray, a wax-based stick or a towelette. Such formulations may beneficially include any ingredient conventionally used in the cosmetics field. These ingredients include preservatives, aqueous phase thickeners, fatty-phase thickeners, fragrances, hydrophilic and lipophilic active agents, as well as pigments, fillers, oils, one or more waxes or gums, or mixtures of any of the foregoing.

In addition, the aforementioned formulations may include one or more of the following: a skin penetration enhancer, a dermal delivery system, an emollient, a skin plumper, an optical diffuser, a sunscreen, an exfoliation promoter and an antioxidant. A dermal delivery system may be liposomes, nanosomes, phosopholipid-based non-liposome compositions (e.g., selected cochleates), among others. Details with respect to these and other suitable cosmetic ingredients can be found in the International Cosmetic Ingredient Dictionary and Handbook (ICID), 10^{th} ed., Cosmetic, Toiletry and Fragrance Association, at 2177-2299 (2004).

These natural products can also be incorporated into a transdermal patch or similar delivery system. The transdermal patch can be of conventional construction, e.g., of the type used to deliver sustained doses of estrogen, nitroglycerine, fentenyl, or the like.

In other embodiments of the invention, the benefits of 3DG-containing natural products for use as food, cosmetic, pharmaceutical or dietary supplement ingredients can be enhanced by purifying or refining processes that reduce the 3DG content thereof. The 3DG concentration of natural products can be determined using the measurement technique described in Example 2, below.

The purification or refining processing contemplated by the present invention involves admixture of the natural product with at least one 3DG inactivating agent. Representative examples of suitable 3DG inactivating agents are listed in Table 3, below. Arginine is a preferred 3DG inactivating agent for use in practicing this embodiment of the invention.

In view of the potential harmful health effects of 3DG, any measurable reduction in the 3DG content of natural products used as food, cosmetic, pharmaceutical, or dietary supplement ingredients will provide a benefit.

This same method can be utilized to reduce the 3DG content of foods, food additives or beverages, such as carbonated beverages, which may be fermented (e.g., beer, ale or the like) or not (e.g., colas), as well as non-carbonated beverages, which may be fermented (e.g., wine) or not (e.g., fruit juice, fruit punch, vegetable juice or tea).

The following methodologies and experimental data are provided to describe the various embodiments of the invention in further detail. These methods and data are provided for illustrative purposes only and should in no way be construed as limiting the invention.

### F3K Assay

Fructosamine-3-kinase (F3K) phosphorylates fructoselysine to form fructoselysine-3-P, which spontaneously decomposes to give lysine, Pi, and 3DG. The assay is performed in a 96-well plate, with each well containing 100 μl of 50 mM Hepes, pH 8.0, 1 mM Mg-ATP, and 0.20 mM fructoselysine (Dynamis Therapeutics). Five μl of test inhibitor sample was added and the reaction initiated with 120 nM human recombinant F3K enzyme (Dynamis Therapeutics). The plate was incubated at 37° C. for 24 hours to allow F3K to produce FL3P and then to decompose releasing Pi and 3DG. 3DG was measured as in Example 2.

### Natural Products and Chemicals

Aqueous extracts were prepared from various commercially available natural products. Concentrations of the resulting extracts are given below on a weight-per-weight basis, unless otherwise indicated. LFK extract and powder is from lysed Enterococcus faecalis FK-23. Fresh fruits and vegetable extracts were made in a juicer machine (Juiceman Automatic Juice Extractor). Strawberry leaf extract (50% w/w in water) was similarly made. Samples were allowed to settle or were centrifuged (12,000×g, 10 min) before removing an aliquot of the supernatant for analysis.

### Example 1

### F3K Inhibition

F3K activity was measured in the presence of various natural product extracts using the above-described assay. The percent inhibition is shown in Tables 1 and 1A. Extracts from chestnut skin, lychee seed, grapeseed, gooseberry, peanut skin, cat's claw and rose inhibited F3K activity by more than 90%.

TABLE 1
F3K Activity in the Presence of Natural Product Extracts

| Sample | Test Conc | (Inhibition %) |
|---|---|---|
| Clam Extract | 0.125% | 43 |
| | 0.25% | 32 |
| Chestnut Skin | 0.1% | 93 |
| Porcine Collagen | 0.125% | 13 |
| | 0.25% | 25 |
| Fish Collagen | 0.125% | 25 |
| | 0.25% | 31 |
| Rose Extract #1 | 0.25% | 98 |
| Rose Extract #2 | 0.25% | 93 |
| LFK Extract | 0.5% | 23 |
| LFK Powder | 0.5% | 12 |
| Plant Extract 2 | 0.5% | 84 |
| Grapeseed Extract | 0.5% | 96 |
| Lychee Seed Extract | 0.5% | 98 |
| | 0.25% | 94 |
| Broccoli Sprout 1 Extract | 0.5% | 20 |
| Broccoli Extract | 0.5% | 0 |
| Broccoli Sprout 2 Extract | 0.5% | 20 |
| Peanut Skin Extract | 0.5% | 96 |
| Herbal Mixture | 0.5% | 69 |
| Indian Gooseberry Extract | 0.5% | 98 |
| Rafuma Extract | 0.5% | 79 |
| Cat's Claw Extract | 0.5% | 95 |

TABLE 1A
Inhibition of F3K enzyme activity by Fresh
Fruit and Vegetable Extracts, Herbs and Teas

| Sample | Test Concentration (%) | Inhibition (%) |
|---|---|---|
| Tomato | 5 | 73 |
| Artichoke | 5 | 92 |
| Artichoke | 0.5 | 61 |
| Broccoli | 5 | 99 |
| Carrot | 5 | 88 |
| Cucumber flesh | 5 | 78 |
| Cucumber skin | 5 | 36 |
| Garlic | 5 | 66 |
| Green Bean | 5 | 30 |
| Green Squash | 5 | 62 |
| Bitter Melon | 5 | 90 |
| Plum | 5 | 96 |
| Plum | 0.5 | 87 |
| Red Grapes | 5 | 91 |
| Green Grapes | 5 | 68 |
| Blueberry | 5 | 55 |
| Blackberry | 5 | 76 |
| Raspberry | 5 | 74 |
| Strawberry | 5 | 86 |
| Peppermint Leaf | 0.25 | 98 |
| Peppermint Leaf | 0.025 | 90 |
| Rose Flower | 0.25 | 98 |
| Rose Flower | 0.025 | 77 |
| Strawberry Leaf | 0.25 | 96 |
| Strawberry Leaf | 0.025 | 75 |
| Ginkgo Leaf | 0.25 | 84 |
| Ginkgo Leaf | 0.025 | 42 |
| Coffee | 0.25 | 73 |
| KeT Chi | 0.25 | 16 |
| Chrysanthemum | 0.25 | 34 |
| Green Tea #1 | 0.12 | 89 |
| Green Tea #2 | 0.12 | 56 |
| Black Tea | 0.12 | 61 |
| Ginseng Tea | 0.12 | 81 |
| Organic Green Tea | 0.12 | 61 |
| cranberry powder | 0.25 | 92 |
| wild cherry bark powder | 0.25 | 94 |
| cat's claw bark powder | 0.25 | 95 |
| comfrey root powder | 0.25 | 82 |
| myrrh gum powder | 0.25 | 56 |
| Tomato powder | 0.25 | 73 |
| Blackberry leaf | 0.25 | 32 |
| Birch Leaf | 0.25 | 3 |
| Birch Bark | 0.25 | 15 |
| Basil Leaf | 0.25 | 60 |
| Artichoke Leaf | 0.25 | 41 |
| Whole Oat tops | 0.25 | 7 |
| Mistletoe | 0.25 | 45 |
| Mandrake/May Apple root | 0.25 | 71 |
| Lemon Balm Herb | 0.25 | 55 |
| Blue Cohosh | 0.25 | 25 |
| Strawberry Leaf | 0.25 | 14 |
| Spearmint Leaf | 0.25 | 39 |
| St John's Wort herb | 0.25 | 35 |
| Raspberry Leaf | 0.25 | 36 |
| Peppermint Leaf | 0.25 | 63 |
| White Willow Bark Powder | 0.025 | 74 |
| Licorice Root Powder | 0.025 | 47 |
| Mustard seed Powder | 0.025 | 43 |
| Ginkgo Leaf Powder | 0.025 | 31 |
| Irish Moss Powder | 0.025 | 29 |

### Example 2

### Endogenous 3DG Concentrations in Natural Product and Chemical Samples

Levels of 3DG in F3K assay samples and various natural products prepared in PBS were measured, using the following technique.

### Measurement of 3DG

### Reagents

### 50 mM Phosphate Buffer pH 7.2 (PBS) (Sigma)

### Ethyl Acetate (Fisher)

### N-methyl-N-(Trimethylsilyl)-triifluoroacetamide (MSTFA) (Acros Organics)

### 2,3-Diamino Naphthalene (DAN) Sigma

10 uM U-¹³C-3-deoxyglucosone (3DG) as an internal standard

### Reagent Preparation

### Reagent 1: 50 mM Phosphate Buffer pH 7.2 (PBS)

### Reagent 2: 0.1 g DAN to 1% in 100 mL PBS

### Reagent 3: 10 uM U-¹³C-3DG

### Reagent 4: Ethyl Acetate

### Reagent 5: N-methyl-N-(Trimethylsilyl)-triifluoroacetamide (MSTFA)

### Equipment

GC-MS: 6850 Automatic Liquid Sampler/G2570A 6850 GC/MSD System/G1701 DA GC/MSD Chem Station Agilent

### Analysis Set Up

1. Combine samples (100 uL-1 mL) with Reagent 1 to a total of 1 mL.
2. Add 1 mL of Reagent 2 and 20 uL of Reagent 3.
3. Vortex and let sit for 10 hours at room temperature.
4. Add 1 mL of Reagent 4 and vortex.
5. Let sit for 5 minutes and add another 1 mL of Reagent 4.
6. Centrifuge about 10 minutes.
7. Move the upper layer to another tube and centrifuge in Speed-Vac about 30 min with vacuum.
8. Add 200 uL of Reagent 4 and vortex.
9. Transfer into another tube for GC-MS
10. Centrifuge about 15 minutes with vacuum in Speed-Vac.
11. Add 100 μL of Reagent 5.
12. Heat at 50° about 60 minutes in a block heater.
13. Analyze by GC-MS for I295 (¹²C-3DG) and I299 (U-¹³C-3DG).

The results are shown in Table 2. Several natural product extracts and glucosamine-hydrochloride contained >100 μM 3DG.

TABLE 2
Levels of 3DG in Various Compositions

| Sample | Concentration | 3DG Conc (uM) |
|---|---|---|
| Chestnut Skin | 0.1% * | 16.30 |
| Rose Extract #1 | 5% * | 33.36 |
| Rose Extract #2 | 5% * | 309.91 |
| LFK Extract | 10% * | 20.75 |
| LFK Powder | 10% * | 20.06 |
| Plant Extract | 10% * | 247.99 |
| Grapeseed Extract | 10% * | 11.63 |
| Lychee Seed Extract | 10% * | 26.50 |
| Glucosamine HCl | 100 mM | 1011.94 |
| Tranexamic Acid | 100 mM | 54.69 |
| Broccoli Sprout Extract | 5% * | 23.13 |
| Broccoli Extract | 5% * | 272.32 |
| Broccoli Sprout Extract | 5% * | 53.71 |
| Peanut Skin Extract | 5% * | 29.31 |
| Herbal Mixture | 5% * | 255.57 |
| Gooseberry Extract | 5% * | 14.58 |
| Rafuma Extract | 5% * | 649.01 |
| Cat's Claw Extract | 5% * | 54.98 |

* denotes samples that contained insoluble material.

### Example 3

### 3DG Inactivation Assay

The following assay was used to determine inactivation of 3DG by various natural products and chemicals.

### 3DG Binding Assay

### Reagents

### Reagent 1: 50 mM Phosphate Buffer pH 7.2 (PBS)

### Reagent 6: 620 μM ¹²C-3DG

### Incubation Set Up

1. Dilute the samples with Reagent 1 to a volume of 1.9 ml and add 100 uL Reagent 6 to a total of 2 mL.
2. Take 600 μL from each solution before incubation (as 0 day sample)
3. Let sit for 24 hours and 72 hours at 37° and remove 600 μL for samples on day 1 and day 3)
4. Measure 3DG levels as in Example 2
   The results are set forth in Table 3. Several chemicals and natural product extracts showed 3DG inactivating activity. Samples with the most amount of 3DG inactivating activity were arginine, clam extract, chestnut skin extract, pig and fish collagens, pyridoxal-5′-phosphate, grapeseed extract, lychee seed extract, peanut skin extract and cat's claw extract. Most of the chestnut skin 3DG inactivating activity was in the supernatant after centrifugation. Some samples showed high intrinsic levels of 3DG including Chitosan L, glucosamine, Rafuma extract, broccoli extract and herb mixture.

TABLE 3

| | | 3DG Conc (uM) | | |
|---|---|---|---|---|
| Sample | Concentration | 0 day | 1 day | 3 days |
| Arginine | 0 mM | | 15.73 | 13.50 |
| | 2 mM | | 15.71 | 13.14 |
| | 6 mM | | 13.66 | 8.50 |
| | 12 mM | | 12.24 | 4.98 |
| Clam Extract | 0% | | 19.09 | 14.99 |
| | 0.5% | | 17.36 | 6.53 |
| | 1.5% | | 16.70 | 9.60 |
| | 2.5% | | 18.18 | 11.94 |
| Chitosan L | 0% | | 19.09 | 14.99 |
| | 0.5% | | 41.18 | 16.07 |
| | 1.5% | | 75.62 | 65.85 |
| | 2.5% | | 103.60 | 101.0 |
| Chestnut Skin | 0% | | 19.09 | 14.99 |
| Extract | 0.1% | | 9.14 | 2.35 |
| | 0.2% | | 5.81 | 1.73 |
| | 0.5% | | 5.81 | 1.66 |
| | 0% | 16.72 | 17.07 | |
| | 0.1% | 16.30 | 7.98 | |
| | 0.1% Supernatant | 16.90 | 8.52 | |
| | 0.1% Pellet | 16.03 | 15.95 | |
| Ascorbic Acid | 0 mM | | 18.58 | 15.50 |
| | 5 mM | | 13.75 | 11.86 |
| | 15 mM | | 13.99 | 10.18 |
| | 30 mM | | 17.26 | 12.64 |
| Thiamine nitrate | 0 mM | | 18.58 | 15.50 |
| | 5 mM | | 15.87 | 16.45 |
| | 15 mM | | 18.90 | 14.19 |
| | 30 mM | | 17.20 | 14.85 |
| Calcium | 0 mM | | 18.58 | 15.50 |
| Panthenate | 2.5 mM | | 17.27 | 16.18 |
| | 7.5 mM | | 16.92 | 15.37 |
| | 12.5 mM | | 18.58 | 14.57 |
| Carnosine | 0 mM | | 18.07 | 16.81 |
| | 5 mM | | 16.18 | 14.58 |
| | 15 mM | | 16.90 | 15.39 |
| | 30 mM | | 18.45 | 13.97 |
| | 0 mM | | 15.73 | 13.50 |
| | 2 mM | | 16.85 | 15.58 |
| | 6 mM | | 15.96 | 16.93 |
| | 12 mM | | 15.05 | 12.37 |
| Pig Collagen | 0% | | 17.42 | 10.26 |
| | 0.25% | | 13.76 | 4.64 |
| | 0.75% | | 14.41 | 3.57 |
| | 1.5% | | 13.30 | 4.06 |
| Fish Collagen | 0% | | 17.42 | 14.54 |
| | 0.25% | | 15.18 | 9.48 |
| | 0.75% | | 14.33 | 9.87 |
| | 1.5% | | 13.82 | 7.41 |
| Meglumine-HCl | 0 mM | | 18.58 | 15.50 |
| | 5 mM | | 17.51 | 16.27 |
| | 15 mM | | 16.58 | 17.52 |
| | 30 mM | | 15.39 | 15.87 |
| Pyridoxal-5′- | 0 mM | | 18.07 | 16.81 |
| Phosphate | 2 mM | | 13.71 | 12.29 |
| | 6 mM | | 13.00 | 8.12 |
| | 12 mM | | 12.20 | 7.14 |
| Thiamine-HCl | 0 mM | | 18.07 | 16.81 |
| | 5 mM | | 16.27 | 14.49 |
| | 15 mM | | 14.83 | 14.81 |
| | 30 mM | | 17.76 | 14.89 |
| Pyridoxine | 0 mM | 16.96 | 17.25 | 16.39 |
| | 2 mM | 16.32 | 14.82 | 16.35 |
| | 6 mM | 16.13 | 16.67 | 15.83 |
| | 12 mM | 15.90 | 15.85 | 15.44 |
| Pyridoxal-HCl | 0 mM | 16.96 | 17.25 | 16.39 |
| | 2 mM | 16.46 | 12.16 | 16.19 |
| | 6 mM | 16.02 | 14.57 | 15.72 |
| | 12 mM | 14.80 | 14.79 | 14.99 |
| Rose Extract-1 | 0% | 16.60 | 16.29 | 16.16 |
| | 0.25% | 12.79 | 4.03 | 0.73 |
| | 0.75% | 12.69 | 11.04 | 2.98 |
| | 1.5% | 18.58 | 20.88 | 13.58 |
| Rose Extract-2 | 0% | 16.60 | 16.29 | 16.16 |
| | 0.25% | 32.93 | 10.59 | 1.04 |
| | 0.75% | 52.66 | 12.73 | 0.95 |
| | 1.5% | 72.17 | 27.79 | 1.81 |
| LFK1 | 0% | 16.60 | 16.29 | 16.16 |
| | 0.5% | | 15.96 | 8.35 |
| | 1.5% | | 20.10 | 11.47 |
| | 3% | | 20.20 | 12.33 |
| LFK2 | 0% | 16.60 | 16.29 | 16.16 |
| | 0.5% | | 17.39 | 6.57 |
| | 1.5% | | 17.90 | 9.74 |
| | 3% | | 22.14 | 12.79 |
| Plant Extract | 0% | 16.60 | 16.29 | 16.16 |
| | 0.5% | 27.32 | 11.55 | 3.26 |
| | 1.5% | 45.95 | 22.65 | 6.88 |
| | 3% | 72.36 | 51.77 | 19.09 |
| Grapeseed Extract | 0% | 16.60 | 16.29 | 16.16 |
| | 0.5% | 15.27 | 4.85 | 0.98 |
| | 1.5% | 15.45 | 5.25 | 1.00 |
| | 3% | 18.30 | 5.77 | 1.66 |
| Lychee Seed | 0% | 16.60 | 16.29 | 16.16 |
| Extract | 0.5% | 17.38 | 10.82 | 2.18 |
| | 1.5% | 18.84 | 7.92 | 2.01 |
| | 3% | 22.76 | 9.93 | 2.96 |
| D-Glucosamine | 0 mM | 21.55 | 20.84 | 17.27 |
| HCl | 5 mM | 25.15 | 43.42 | 82.33 |
| | 15 mM | 36.00 | 122.14 | 181.09 |
| | 30 mM | 56.87 | 183.92 | 173.60 |
| Tranexamic Acid | 0 mM | 21.55 | 20.84 | 17.27 |
| | 5 mM | 20.96 | 24.16 | 22.02 |
| | 15 mM | 18.06 | 18.98 | 19.21 |
| | 30 mM | 19.25 | 17.42 | 16.87 |
| Calcium | 0 mM | 21.55 | 20.84 | 17.27 |
| Panthenate | 5 mM | 21.06 | 18.13 | 17.96 |
| | 15 mM | 22.86 | 18.84 | 16.95 |
| | 30 mM | 22.09 | 19.56 | 19.17 |
| Panthenol | 0 mM | 21.55 | 20.84 | 17.27 |
| | 5 mM | 19.54 | 18.10 | 19.51 |
| | 15 mM | 18.08 | 17.74 | 16.84 |
| | 30 mM | 18.91 | 17.67 | 17.63 |
| Broccoli Sprout | 0% | 21.55 | 20.84 | 17.27 |
| Extract 1 | 0.5% | 20.25 | 17.38 | 14.68 |
| | 1.5% | 24.88 | 21.97 | 16.62 |
| | 3.0% | 25.29 | 22.35 | 17.90 |
| Broccoli Extract | 0% | 21.55 | 20.84 | 17.27 |
| | 0.5% | 37.06 | 37.22 | 28.38 |
| | 1.5% | 76.62 | 71.39 | 34.15 |
| | 3.0% | 108.33 | 108.79 | 33.24 |
| Broccoli Sprout | 0% | 21.55 | 20.84 | 17.27 |
| Extract 2 | 0.5% | 22.40 | 19.08 | 18.20 |
| | 1.5% | 28.67 | 24.12 | 19.77 |
| | 3.0% | 36.55 | 29.75 | 21.48 |
| Peanut Skin Extract | 0% | 21.55 | 20.84 | 17.27 |
| | 0.5% | 21.27 | 5.09 | 2.18 |
| | 1.5% | 24.37 | 7.00 | 2.63 |
| | 3.0% | 25.02 | 9.76 | 3.79 |
| Herb Mixture | 0% | 21.55 | 20.84 | 17.27 |
| | 0.5% | 33.57 | 19.43 | 16.68 |
| | 1.5% | 62.01 | 24.00 | 13.76 |
| | 3.0% | 92.87 | 47.96 | 20.03 |
| Gooseberry Extract | 0% | 21.55 | 20.84 | 17.27 |
| | 0.5% | 20.64 | 2.13 | 0.98 |
| | 1.5% | 21.92 | 4.27 | 1.89 |
| | 3.0% | 25.18 | 10.13 | 3.69 |
| Rafuma Extract | 0% | 21.55 | 20.84 | 17.27 |
| | 0.5% | 78.58 | 39.03 | 5.89 |
| | 1.5% | 135.81 | 119.92 | 82.19 |
| | 3.0% | 174.07 | 166.79 | 165.95 |
| Cat's Claw Extract | 0% | 21.55 | 20.84 | 17.27 |
| | 0.5% | 22.69 | 8.22 | 2.94 |
| | 1.5% | 28.65 | 11.23 | 4.93 |
| | 3.0% | 38.58 | 17.58 | 8.35 |

### Example 4

### 3DG Levels in Beverages and Foods

3DG levels were measured in various brand name beverages and foods; results are shown in Table 4. Miso soup, soy sauce and all non-alcoholic beverages except diet soda and one brand of Green Tea contain high levels of 3DG (>50 μM). All beers contain >300 μM 3DG and dark beers contain the highest levels of 3DG (>600 μM). Plum wine contained high levels of 3DG and red wine had relatively low levels of 3DG.

TABLE 4

| | Country of | |
|---|---|---|
| Sample | Origin | μM 3DG |
| Cola soda A | Japan | 286.59 |
| Cola soda B | US | 491.58 |
| Cola soda C | US | 550.28 |
| Diet Cola soda A | US | 6.62 |
| Diet Cola soda B | US | 10.64 |
| Lemon-Lime Drink | US | 159.81 |
| Fruit Punch Drink | US | 55.71 |
| Lemonade | US | 499.06 |
| Orange Juice | US | 87.14 |
| Grape Juice | US | 701.54 |
| Vegetable Juice | US | 321.51 |
| Green Tea A | US | 608.88 |
| Green Tea B | US | 246.68 |
| Green Tea C | Japan | 1.14 |
| Beer A | Japan | 420.47 |
| Beer B | Japan | 329.46 |
| Beer C | Japan | 529.46 |
| Beer D | Japan | 463.94 |
| Beer E | US | 370.06 |
| Beer F | US | 759.09 |
| Beer G | US | 322.48 |
| Beer H | Germany | 360.79 |
| Dark Beer A | Japan | 757.37 |
| Dark Beer B | Ireland | 646.63 |
| Red Wine | US | 125.98 |
| Japanese Syotyu | Japan | 2.84 |
| Plum Wine | Japan | 1582.25 |
| Soy Sauce | Japan | 979.35 |
| Miso (5% Solution) | Japan | 745.65 |

A number of patent and non-patent publications are cited in the foregoing specification in order to describe the state of the art to which this invention pertains. The entire disclosure of each of these publications is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims. Furthermore, the transitional terms “comprising”, “consisting essentially of” and “consisting of” define the scope of the appended claims, in original and amended form, with respect to what unrecited additional claim elements or steps, if any, are excluded from the scope of the claims. The term “comprising” is intended to be inclusive or open-ended and does not exclude additional, unrecited elements, methods step or materials. The phrase “consisting of” excludes any element, step or material other than those specified in the claim, and, in the latter instance, impurities ordinarily associated with the specified materials. The phrase “consisting essentially of” limits the scope of a claim to the specified elements, steps or materials and those that do not materially affect the basic and novel characteristic(s) of the claimed invention. All compositions or formulations identified herein can, in alternate embodiments, be more specifically defined by any of the transitional phases “comprising”, “consisting essentially of” and “consisting of”.

What is claimed is:

1. A method for the treatment or prophylaxis of a condition or disease state which is alleviated by inhibiting the enzymatic conversion of fructoselysine to fructoselysine-3-phosphate, in a patient in need of said treatment or prophylaxis, said method comprising administering to said patient at least one natural product having as a component thereof an inhibitor of said enzymatic conversion of fructoselysine to fructoselysine-3-phosphate, in an amount effective to inhibit said conversion.

2. The method of claim 1, wherein oxidative stress contributes to the onset and/or progression of said condition or disease state.

3. The method of claim 2, wherein the production of advanced glycation end products (AGEs) contributes to the onset and/or progression of said condition or disease state.

4. The method of claim 1, wherein said disease state is selected from the group consisting of inflammatory disorders, diabetes complications, diseases of aging, hypertension, stroke, neurodegenerative disorders, circulatory disease, atherosclerosis, osteoarthritis and cataracts.

5. The method of claim 1, wherein said at least one natural product administered to said patient is from the group of natural products set forth in Tables 1 and 1A of the foregoing specification.

6. The method of claim 1, wherein said patient is a human.

7. The method of claim 1, wherein said patient is a domestic animal.

8. The method of claim 1, wherein said condition is a skin condition.

9. The method of claim 8, wherein said skin condition is associated with intrinsic or extrinsic aging.

10. The method of claim 8 or 9, wherein symptoms of said skin condition include at least one of wrinkling in the skin, fine lines in the skin, inelasticity of the skin, coarse texture and uneven pigmentation of the skin.

11. A method of preventing, ameliorating and/or reversing the intrinsic and/or extrinsic aging of skin, said method comprising topically applying to said aging skin a composition comprising at least one natural product having as a component thereof an inhibitor of the enzymatic conversion of fructoselysine to fructoselysine-3-phosphate, in an amount effective to inhibit said conversion.

12. The method of claim 11, wherein said at least one natural product is from the group of natural products set forth in Tables 1 and 1A of the foregoing specification.

13. A method of improving the appearance, texture, or elasticity of aging skin, said method comprising topically applying to said aging skin a composition comprising at least one natural product having as a component thereof an inhibitor of the enzymatic conversion of fructoselysine to fructoselysine-3-phosphate, in an amount effective to inhibit said conversion.

14. The method of claim 13, wherein said at least one natural product is from the group of natural products set forth in Tables 1 and 1A of the foregoing specification.

15. A method of treating skin damage due to oxidative stress and/or the production of AGEs, said method comprising topically applying to said damaged skin a composition comprising a natural product having as a component thereof an inhibitor of the enzymatic conversion of fructose-lysine to fructose-lysine-3-phosphate, in an amount effective to inhibit said conversion.

16. The method of claim 15, wherein said at least one natural product is from the group of natural products set forth in Tables 1 and 1A of the foregoing specification.

17. A method of enhancing the purity of preparations containing glucosamine and its pharmaceutically acceptable salts and derivatives as a food, cosmetic, pharmaceutical or dietary supplement ingredient, said method comprising reducing the 3-deoxyglucosone (3DG) content thereof.

18. The method of claim 17, wherein said 3DG content is reduced by admixture with a 3DG inactivating agent.

19. The method of claim 18, wherein said 3DG inactivating agent is selected from the group consisting of arginine, chestnut skin extract, collagen, pyridoxal 5′-phosphate, rose extract, LFK1, LFK2, plant extract, grapeseed extract, lychee seed extract, peanut skin extract, gooseberry extract, Rafuma extract and cat's claw extract.

20. The method of claim 18, wherein said 3DG inactivating agent is arginine.

21. A method of refining a 3DG-containing natural product as a food, cosmetic, pharmaceutical or dietary supplement ingredient, said method comprising reducing the 3DG content of said natural product.

22. The method of claim 21, wherein said 3DG content is reduced by admixture with a 3DG inactivating agent.

23. The method of claim 22, wherein said 3DG inactivating agent is selected from the group consisting of arginine, chestnut skin extract, collagen, pyridoxal 5′-phosphate, rose extract, LFK1, LFK2, plant extract, grapeseed extract, lychee seed extract, peanut skin extract, gooseberry extract, Rafuma extract and cat's claw extract.

24. The method of claim 22, wherein said 3DG inactivating agent is arginine.

25. A method of reducing the 3-deoxyglucosone (3DG) content of a 3DG-containing beverage, food or food additive, said method comprising admixing a 3DG inactivator with said beverage, food or food additive.

26. The method of claim 25, wherein said 3DG inactivating agent is selected from the group consisting of arginine, chestnut skin extract, collagen, pyridoxal 5′-phosphate, rose extract, LFK1, LFK2, plant extract, grapeseed extract, lychee seed extract, peanut skin extract, gooseberry extract, Rafuma extract and cat's claw extract.

27. The method of claim 25, wherein said 3DG inactivating agent is arginine.

28. The method of claim 25, wherein said 3DG inactivating agent is admixed with a carbonated beverage.

29. The method of claim 28, wherein said carbonated beverage is a fermented beverage.

30. The method of claim 25, wherein said 3DG is admixed with a non-carbonated beverage.

31. The method of claim 30, wherein said beverage is a fermented beverage.

32. The method of claim 30, wherein said beverage is a fruit juice, fruit punch, vegetable juice or a tea.

33. A method for treatment or prophylaxis of a dry mucus membrane disorder, the method comprising administering to a patient in need of said treatment or prophylaxis a natural product that increases goblet cell proliferation, in an amount effective to cause said proliferation.

34. The method of claim 33, wherein said disorder is selected from the group of dry eye, dry mouth and constipation.

35. A topical composition comprising at least one natural product comprising an agent that acts to inhibit the enzymatic production of 3-deoxyglucosone from fructoselysine, and at least one dermatologically acceptable excipient.

36. The composition of claim 35, further comprising at least one of a skin penetration enhancer, an emollient, a skin plumper, an optical diffuser, a sunscreen, an exfoliation promoter and an antioxidant.

37. The composition of claim 1, wherein said excipient is a delivery vehicle comprising at least one of liposomes, nanosomes and phosopholipid-based non-liposome formulations.

38. An article of manufacture comprising the composition of claim 35 incorporated into a transdermal patch.

39. A topical composition comprising at least one natural product comprising an inactivator of 3-deoxyglucosone, and at least one dermatologically acceptable excipient.

40. The composition of claim 39, wherein said excipient is selected from the group of a skin penetration enhancer, an emollient, a skin plumper, an optical diffuser, a sunscreen, an exfoliation promoter and an antioxidant.

41. The composition of claim 1, including a delivery vehicle comprising liposomes, nanosomes and phosopholipid-based non-liposome formulations.

42. An article of manufacture comprising the composition of claim 35 incorporated into a transdermal patch.
